(19) United States
(12) Patent Application Publication
(10) Pub. No.: US 2007/0293463 A1
(43) Pub. Date: Dec. 20, 2007

(54) METHOD FOR SHORTENING HOSPITAL STAY IN PATIENTS WITH CONGESTIVE HEART FAILURE AND ACUTE FLUID OVERLOAD

(76) Inventors: Howard Dittrich, San Diego, CA (US); Brian Farmer, San Diego, CA (US); Randy Woods, Rancho Santa Fe, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/763,993

(22) Filed: Jun. 15, 2007

Related U.S. Application Data
(60) Provisional application No. 60/814,109, filed on Jun. 16, 2006.

(51) Int. Cl. A61K 31/58; A61K 31/549; A61K 31/4747; A61K 31/522

(52) U.S. Cl. 514/172; 514/223.5; 514/278; 514/263.32

(57) ABSTRACT

Methods of treating patients with acute fluid overload comprising administering diuretic therapy and an amount of KW-3902, a pharmaceutically acceptable salt, ester, amide, metabolite, or prodrug thereof, effective to accelerate removal of excess fluid from the patient in comparison to diuretic therapy alone. Methods of improving the treatment time to achieve adequate diuresis in an individual experiencing acute fluid overload comprising administering to said individual a diuretic and a therapeutically effective amount of KW-3902 or a pharmaceutically acceptable salt, ester, amide, metabolite, or prodrug thereof.

### RELATED APPLICATIONS

The present application claims priority to U.S. Provisional Application Ser. No. 60/814,109, filed on Jun. 16, 2006, by Dittrich et al. and entitled “METHOD FOR SHORTENING HOSPITAL STAY IN PATIENTS WITH CONGESTIVE HEART FAILURE AND ACUTE FLUID OVERLOAD,” which is hereby expressly incorporated by reference in its entirety.

### BACKGROUND OF THE INVENTION

1. Field of the Invention

The present invention relates to xanthine derivatives and to their use in the treatment of patients with acute fluid overload.

2. Description of the Related Art

Fluid overload is a condition in which an excess of fluid exists in the circulation. The leading cause of fluid overload is congestive heart failure (CHF). Advanced CHF accounts for over 1 million hospital admissions yearly in the United States (U.S.) and is associated with a 5-year mortality rate of 40%-50%. (American Heart Association 2001 Heart and Stroke Statistical Update, Dallas, Tex.; American Heart Association, 2000; Massie, B. M., and Shah, N. B., Am. Heart J. 133:703-712 (1997)). Currently, the vast majority of HF patients presenting with acute fluid overload are treated with IV diuretics, inotropes and combination therapies. These pharmacological approaches may not always provide timely relief of symptoms without adverse effects for those with advanced HF. Patients manifest signs of persistent fluid overload for an average of 15 hours after admission and readmission rates are 30-60% within 6 months of initial discharge. In the U.S., CHF is currently the most costly cardiovascular disease, with the total estimated direct and indirect costs approaching $56 billion in 1999. (Rich, M. W., and Nease, R. F., Arch Intern Med 159-1690-1700 (1999)). The estimated cost of a single hospital admission primarily for CHF is $11,000 (Massie, B. M., and Shah, N. B., Am. Heart J. 133:703-712 (1997); Rich, M. W., and Nease, R. F., Arch Intern Med 159:1690-1700 (1999)), whereas the Medicare Diagnostic-Related Group (DRG) reimbursement is approximately $4,300. This represents an increased economic burden for hospitals, as Medicare is the primary provider for this disease.

Efforts to contain rising costs for hospital acute care have resulted in shorter hospital stays for patients with acutely decompensated CHF. However, these shorter stays may not allow for adequate diuresis. There is a need for therapies that provide adequate diuresis with reduced intravenous diuretic therapy. Such therapies would reduce the length of hospital stay and thus the costs associated with treatment.

### BRIEF SUMMARY OF THE INVENTION

Provided herein are methods for treating individuals with acute fluid overload. In one embodiment, a patient in need of short-term hospitalization to treat acute fluid overload can be identified, the patient can be hospitalized, and administered intravenous diuretic therapy and an amount of KW-3902 or a pharmaceutically acceptable salt, ester, amide, metabolite, or prodrug thereof, effective to accelerate removal of excess fluid from the patient compared to diuretic therapy alone.

In some embodiments, the administration of KW-3902 or pharmaceutically acceptable salt, ester, amide, metabolite or prodrug thereof is effective to reduce the amount of intravenous diuretic therapy necessary to achieve adequate diuresis.

In some embodiments, the daily dose of the intravenous diuretic is decreased over time.

In some embodiments, the administration of KW-3902 or pharmaceutically acceptable salt, ester, amide, metabolite or prodrug thereof is effective to reduce the term of the short-term hospitalization.

In some embodiments, the diuretic can be a proximal diuretic, a distal diuretic, or a loop diuretic. For example, the diuretic can be selected from hydrochlorazides, fluorosemide, torsemide, bumetanide, ethacrynic acid, piretanide, spironolactone, triamterene, and amiloridehiazides. Preferably, the diuretic is furosemide.

In some embodiments, the methods disclosed herein are used to treat a patient that has congestive heart failure.

In some embodiments, the amount of KW-3902 or pharmaceutically acceptable salt, ester, amide, metabolite, or prodrug thereof can be effective to improve renal function, as measured by creatinine clearance rate. For example, in some embodiments, the amount of KW-3902 or pharmaceutically acceptable salt ester, amide, metabolite, or prodrug thereof is administered to the patient in daily doses of about 2.5 mg, 5 mg, 10 mg, 15 mg, 20 mg, 25 mg, 30 mg, 35 mg, 40 mg, 45 mg, 50 mg, 55 mg, 60 mg, or 70 mg. Preferably, the amount of KW-3902 or pharmaceutically acceptable salt, ester, amide, metabolite, or prodrug thereof is administered to the individual in daily doses of 30 mg.

In some embodiments, the individual can exhibit a creatinine clearance rate of about 20 to about 80 mL/min.

Some embodiments provide a method of improving the treatment time to achieve adequate diuresis in an individual experiencing acute fluid overload. The method can include the steps of identifying an individual in need of hospitalization for intravenous diuretic treatment, hospitalizing the individual and administering to the individual intravenous diuretic therapy and a therapeutically effective amount of KW-3902, or a pharmaceutically acceptable salt, ester, amide, metabolite, or prodrug thereof.

In some embodiments, the daily dose of the intravenous diuretic is decreased over time.

In some embodiments, the diuretic can be a proximal diuretic, a distal diuretic, or a loop diuretic. For example, the diuretic can be selected from hydrochlorazides, furosemide, torsemide, bumetanide, ethacrynic acid, piretanide, spironolactone, triamterene, and amiloridehiazides. Preferably, the diuretic is furosemide.

In some embodiments, the methods disclosed herein are used to treat a patient that has congestive heart failure.

In some embodiments, the amount of KW-3902 or pharmaceutically acceptable salt, ester, amide, metabolite, or prodrug thereof can be effective to improve renal function, as measured by creatinine clearance rate. For example, in some embodiments, the amount of KW-3902 or pharmaceutically acceptable salt, ester, amide, metabolite, or prodrug thereof is administered to the patient in daily doses of about 2.5 mg, 5 mg, 10 mg, 15 mg, 20 mg, 25 mg, 30 mg, 35 mg, 40 mg, 45 mg, 50 mg, 55 mg, 60 mg, or 70 mg. Preferably, the amount of KW-3902 or pharmaceutically acceptable salt, ester, amide, metabolite, or prodrug thereof is administered to the individual in daily doses of 30 mg.

### BRIEF DESCRIPTION OF THE DRAWINGS

FIG. 1 shows the urine volume following treatment with placebo or with KW-3902 in individuals with acute fluid overload.

FIG. 2 shows the total dose of IV furosemide following treatment with placebo or with KW-3902 in individuals with acute fluid overload.

### DETAILED DESCRIPTION OF THE INVENTION

Provided herein are methods for treating individuals with acute fluid overload. In one embodiment, a patient in need of short-term hospitalization to treat acute fluid overload can be identified, the patient can be hospitalized, and administered intravenous diuretic therapy and an amount of KW-3902 or a pharmaceutically acceptable salt, ester, amide, metabolite, or prodrug thereof effective to accelerate removal of excess fluid from the patient compared to diuretic therapy alone.

Also provided herein are methods of improving the treatment time to achieve adequate diuresis in an individual experiencing acute fluid overload. The method can include the steps of identifying an individual in need of hospitalization for intravenous diuretic treatment, hospitalizing the individual and administering to the individual intravenous diuretic therapy and a therapeutically effective amount of KW-3902, or a pharmaceutically acceptable salt, ester, amide, metabolite, or prodrug thereof.

Fluid overload is a condition in which water intake exceeds water leaving the body. This causes fluids (mainly water) to build up in various locations in the body and leads to an increase in weight, swelling in the legs and arms (peripheral edema), and/or in the abdomen (ascites). Eventually, this fluid enters the air spaces in the lungs, reduces the amount of oxygen that can enter the blood, and causes shortness of breath (dyspnea). Fluid can also collect in the lungs when an individual is prostrate, rendering nighttime breathing and sleeping difficult (nocturnal dypsnea). Many health conditions such as kidney failure, post-surgical overload and metabolic diseases such as glucose intolerance, hyperglycemia and acid maltase can cause fluid-overload, however congestive heart failure (CHF) is the leading cause. Acute fluid overload can be diagnosed using conventional methods and refers to a sudden increase in fluids in the body.

A characteristic manifestation of CHF is fluid retention and fluid accumulation that causes congestion of the lungs, liver, intestines and peripheral compartments. Signs and symptoms include shortness of breath (dyspnea) fatigue, orthopnea, rales, pitting edema, elevated central venous pressure, pulmonary congestion, weight gain, volume overload, and elevated filling pressures.

As used herein, the term “treatment” or “treating” or any variation thereof does not necessarily refer to a total cure. Any alleviation of any undesired signed or symptoms of the disease or condition to be treated to any extent or the slowing down of the progress of the disease or condition to be treated can be considered treatment. Furthermore, treatment may include acts that may worsen the patient's overall feeling of well being or appearance. Treatment may also include lengthening the life of the patient, even if the symptoms are not alleviated, the disease conditions are not ameliorated, or the patient's overall feeling of well being is not improved. For example, in some embodiments of the methods described herein, increasing urine output volume may be considered treatment, even if the patient is not cured or does not generally feel better.

The term “patient” or “individual” as used herein refers to is a vertebrate, preferably a mammal, more preferably a human. “Mammal” refers to any animal classified as a mammal, including humans, domestic and farm animals, and zoo, sport, or pet animals, such as, for example, horses, sheep, cows, pigs, dogs, cats, etc. Preferably, the mammal is human.

Acute fluid overload is often treated with intravenous diuretics, inotropes and combination therapies, requiring hospitalization. As used herein, the phrase “short term hospitalization” refers to a length of stay (LOS) in a hospital of about 1 to 15 days, for example from about 3 to 12 days, or from about 5 to 10 days.

In some methods provided herein, patients in need of short-term hospitalization to treat acute fluid overload can be identified using standard clinical diagnostic procedures. Non-limiting factors that are commonly evaluated in determining whether an individual requires hospitalization for acute fluid overload include pitting edema (2+) of lower extremities; jugular venous distension; pulmonary edema or pleural effusion; ascites; paroxysmal nocturnal dyspnea or 2-pillow orthopnea.

In some embodiments, the patient suffers from congestive heart failure (CHF). Congestive heart failure (CHF; cardiac failure) is a condition in which weakened heart function exists together with a build-up of body fluid. CHF often occurs when cardiac output is insufficient to meet metabolic demands of the body, or when the heart cannot meet the demands of operating at increased levels of filling/diastolic pressure.

Common causes of congestive heart failure include: narrowing of the arteries supplying blood to the heart muscle (coronary heart disease); prior heart attack (myocardial infarction) resulting in scar tissue large enough to interfere with normal function of the heart; high blood pressure; heart valve disease due to past rheumatic fever or an abnormality present at birth; primary disease of the heart muscle itself (cardiomyopathy); defects in the heart present at birth (congenital heart disease) and infection of the heart valves and/or muscle itself (endocarditis and/or myocarditis). Each of these disease processes can lead to congestive heart failure by reducing the strength of the heart muscle contraction, by limiting the ability of the heart's pumping chambers to fill with blood due to mechanical problems or impaired diastolic relaxation, or by filling the heart's chambers with too much blood.

Advanced congestive heart failure (CHF) includes both acute and chronic presentations. In some embodiments, individuals identified in the methods provided herein have acute congestive heart failure. In other embodiments, individuals in the methods provided herein have chronic congestive heart failure. Patients presenting with acute decompensated CHF can have an acute injury to the heart, such as a myocardial infarction, mitral regurgitation or ventricular septal rupture. Typically, the injury compromises myocardial performance (for example, a myocardial infarction) or valvular/chamber integrity (for example, mitral regurgitation or ventricular septal rupture). Such injuries can result in an acute rise in the left ventricular (LV) filing pressures. The rise in the LV filing pressures results in pulmonary edema and dyspnea. Currently, the vast majority of HF patients are treated with IV diuretics, inotropes and combination therapies.

In some embodiments, the patients identified herein can exhibit impaired renal function. Renal function refers to the ability the kidney to excrete waste and maintain a proper chemical balance. Typically, renal function is measured by plasma concentrations of creatinine, urea, and electrolytes to determine renal function. Creatinine is a byproduct of normal muscle metabolism that is produced at a fairly constant rate in the body and normally filtered by the kidneys and excreted in the urine. It will be appreciated that any method known to those skilled in the art for measuring renal function can be used in the methods described herein. For example, serum creatinine levels, urine creatinine levels, glomerular filtration rate (GFR) and renal plasma flow (RPF) can be used to assess renal function.

In some embodiments, patients exhibit a GFR of less than about 80 mL/min, for example about 20 mL/min, 30 mL/min, 40 mL/min, 50 mL/min, 60 mL/min 70 mL/min or 75 mL/min, or any number in between prior to hospitalization. Accordingly, in some embodiments, the patient exhibits mildly impaired renal function (e.g., a GFR of about 50 to about 80 mL/min). In some embodiments, the patient exhibits moderately impaired renal function (e.g., a GFR of about 30 mL/min to about 50 mL/min). In yet other embodiments, the patient exhibits severely impaired renal function (e.g., a GFR of about 0 mL/min to about 30 mL/min).

Once identified, patients in need of treatment for acute fluid overload can be hospitalized, and intravenous diuretic therapy can be administered to the patient.

Patients in need of intravenous diuretic treatment can be identified using conventional diagnostic methods. For example, an individual in need of IV diuretic treatment can refer to an individual exhibiting one or more signs or symptoms of CHF, e.g., congestion of the lungs, liver, intestines and peripheral compartments, shortness of breath (dyspnea) fatigue, orthopnea, rates, pitting edema, elevated central venous pressure, pulmonary congestion, weight gain, volume overload, and elevated filling pressures, that cannot be managed taking oral therapy, such as oral dosage forms of diuretics.

Diuretics are compounds that elevate the rate of bodily urine excretion (diuresis). Diuretics can also decrease the extracellular fluid (ECF) volume, and are primarily used to produce a negative extracellular fluid balance. Diuretics function by interfering with sodium and water re-absorption in the nephrons. In general, they increase the rate of sodium excretion from the body, thereby decreasing the volume of the ECF. The increase in sodium excretion restores salt homeostasis and lower tonicity, translating into lower blood pressure. The excretion of salt is usually accompanied by the loss of a proportional amount of water.

Individual diuretics act on a specific segment of nephrons, e.g., the proximal tubule, loop of Henle, or distal tubule. Thus, for example, a loop diuretic inhibits re-absorption in the loop of Henle. As a consequence, higher concentrations of sodium are passed downstream to the distal tubule. This initially results in a greater volume of urine, hence the diuretic effect. However, the distal portion of the tubule recognizes the increase in sodium concentration and the kidney reacts in two ways; one is to increase sodium re-absorption elsewhere in the nephron; the other is to feedback via adenosine A₁ receptors to the afferent arteriole where vasoconstriction occurs. This feedback mechanism is known as tubuloglomerular feedback (TGF). This vasoconstriction results in decreased renal blood flow and decreased glomerular filtration rate (GFR). With time, these two mechanisms result in a decrease in diuretic effect and worsening of renal function. This sequence of events contributes to the progression of disease.

Diuretics fall into four classes depending on their mode and locus of action: carbonic anhydrase inhibitors such as acetazolamide inhibit the absorption of NaHCO₃ and NaCl in the proximal tubule; loop diuretics such as furosemide act on the loop of Henle by inhibiting the Na⁺/K+/2Cl⁻ transporter; thiazide type diuretics which inhibit Na⁺/Cl⁻ co-transporters in the distal tubule; and potassium sparing diuretics act on the collecting duct, and decrease the sodium absorption while sparing K⁺ (i.e., as opposed to the other three categories that promote loss of potassium).

In preferred embodiments, the diuretic is non-adenosine modifying diuretic. In some embodiments, the non-adenosine modifying diuretic is a proximal diuretic, i.e., a diuretic that principally acts on the proximal tubule. Examples of proximal diuretics useful in the methods described herein include, but are not limited to, acetazolamide, methazolamide, dichlorphenamide, and carbonic anhydrase inhibitors.

In other embodiments, the non-adenosine modifying diuretic is a loop diuretic, i.e., a diuretic that principally acts on the loop of Henle. Examples of loop diuretics useful in the methods described herein include, but are not limited to, furosemide (LASIX®, bumetanide (BUMEX®), and torsemide (TOREM®).

In yet other embodiments, the non-adenosine modifying diuretic is a distal diuretic, i.e., a diuretic that principally acts on the distal nephron. Examples of distal diuretics useful in the methods described herein include, but are not limited to, metolazone, thiazides and amiloride.

Some diuretics function by affecting adenosine receptors. Adenosine is an intracellular and extracellular messenger generated by all cells in the body. It is also generated extracellularly by enzymatic conversion. Adenosine binds to and activates seven transmembrane g-protein coupled receptors, eliciting a variety of physiological responses. Adenosine itself, substances that mimic the actions of adenosine (agonists), and substances that antagonize its actions have important clinical applications. Adenosine receptors are divided into four known subtypes (i.e., A₁, A₂ₐ, A_{2b}, and A₃). These subtypes elicit unique and sometimes opposing effects. Activation of the adenosine A₁ receptor, for example, elicits an increase in renal vascular resistance while activation of the adenosine A₂ₐ receptor elicits a decrease in renal vascular resistance. In most organ systems, periods of metabolic stress result in significant increases in the concentration of adenosine in the tissue. The heart, for instance, produces and releases adenosine to mediate adaptive responses to stress, such as reductions in heart rate and coronary vasodilatation. Likewise, adenosine concentrations in kidneys increase in response to hypoxia, metabolic stress and many nephrotoxic substances. The kidneys also produce adenosine constitutively. The kidneys adjust the amount of constitutively produced adenosine in order to regulate glomerular filtration and electrolyte reabsorption. Regarding control of glomerular filtration, activation of A₁ receptors leads to constriction of afferent arterioles while activation of A₂ₐ receptors leads to dilatation of efferent arterioles. Activation of A₂ₐ receptors may also exert vasodilatory effects on the afferent arteriole. Overall, the effect of activation of these glomerular adenosine receptors is to reduce glomerular filtration rate. In addition, A₁ adenosine receptors are located in the proximal tubule and distal tubular sites. Activation of these receptors stimulates sodium reabsorption from the tubular lumen. Accordingly, blocking the effects of adenosine on these receptors will produce a rise in glomerular filtration rate and an increase in sodium excretion.

In addition to diuretic therapy, the individual can be administered an amount of an adenosine A₁ receptor antagonist. Preferably, the individual receiving diuretic therapy is administered an amount of KW-3902 or a pharmaceutically acceptable salt, ester, amide, metabolite, or prodrug thereof, effective to accelerate removal of excess fluid from the patient in comparison to the diuretic therapy alone.

KW-3902 is a xanthine-derived adenosine A₁ receptor antagonist (AA₁RA). Its chemical name is 8-(3-noradamantyl)-1,3-dipropylxanthine, also known as 3,7-dihydro-1,3-dipropyl-8-(3-tricyclo[3.3.1.0^{3,7}]nonyl)-1H-purine-2,6-dione, and its structure is

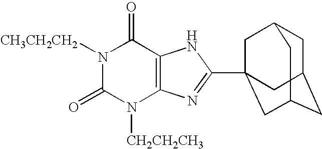

### KW-3902

KW-3902 and related compounds useful in the practice of the present invention are described, for example, in U.S. Pat. Nos. 5,290,782, 5,395,836, 5,446,046, 5,631,260, 5,736,528, 6,210,687, and 6,254,889, the entire disclosure of all of which are hereby incorporated by reference herein, including any drawings.

It will be appreciated that the term “KW-3902” is meant to refer to KW-3902, as well as any pharmaceutically acceptable salt, ester, amide, metabolite, or prodrug thereof.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

The term “metabolite” refers to a compound to which KW-3902 is converted within the cells of a mammal. The pharmaceutical compositions of the present invention may include a metabolite of KW-3902 instead of KW-3902. The scope of the methods of the present invention includes those instances where KW-3902 is administered to the patient, yet the metabolite is the bioactive entity.

Several metabolites of KW-3902 are known and are useful in the methods disclosed herein. These include compounds where the propyl groups on the xanthine entity are hydroxylated, or that the propyl group is an acetylmethyl (CH₃C(O)CH₂—) group. Other metabolites include those in which the noradamantyl group is hydroxylated (i.e., is substituted with a —OH group) or oxylated (i.e., is substituted with a=0 group). Thus, examples of metabolites of KW-3902 include, but are not limited to, 8-(trans-9-hydroxy-3-tricyclo[3.3.1.0^{3,7}]nonyl)-1,3-dipropylxanthine (also referred to herein as “M1-trans”), 8-(cis-9-hydroxy-3-tricyclo[3.3.1.0^{3,7}]nonyl)-1,3-dipropylxanthine (also referred to herein as “M1-cis”), 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine. KW-3902 metabolites used in the pharmaceutical compositions or methods disclosed herein may be a xanthine-derivative compound. The xanthine-derivative compound may be a compound of Formula I or a pharmaceutically acceptable salt thereof, 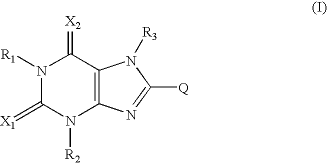
where

each of X₁ and X₂ independently represents oxygen or sulfur;

Q represents: 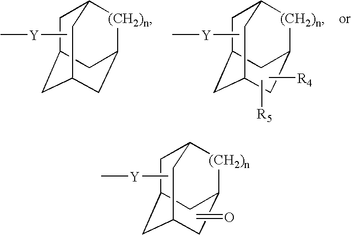
where Y represents a single bond or alkylene having 1 to 4 carbon atoms, n represents 0 or 1;

each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or

R₄ and R₅ are the same or different and each represent hydrogen or hydroxy, and when both R₄ and R₅ are hydrogen, at least one of R₁ and R₂ is hydroxy-substituted or oxo-substituted lower alkyl,
provided that when Q is 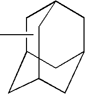
then R₁, R₂ and R₃ are not simultaneously methyl.

In some embodiments, both of R₁ and R₂ of the compound of Formula I are lower alkyl and R₃ is hydrogen; and both of X₁ and X₂ are oxygen. In other embodiments, R₁, R₂ and R₃ independently represent hydrogen or lower alkyl. In still other embodiments, each of R₁ and R₂ independently represents allyl or propargyl and R₃ represents hydrogen or lower alkyl. In certain embodiments, X₁ and X₂ are both oxygen and n is 0.

In some embodiments, R₁ is hydroxy-substituted, oxo-substituted or unsubstituted propyl; R₂ is hydroxy-substituted or unsubstituted propyl; and Y is a single bond. In other embodiments, R is propyl, 2-hydroxypropyl, 2-oxopropyl or 3-oxopropyl; R₂ is propyl, 2-hydroxypropyl or 3-hydroxypropyl.

In some embodiments Q is 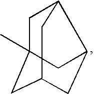
while in other embodiments Q is 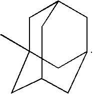
In other embodiments, Q is 9-hydroxy, 9-oxo or 6-hydroxy substituted 3-tricyclo[3.3.1.0^{3,7}]nonyl, or 3-hydroxy-1tricyclo[3.3.1.1.^{3,7}]decyl.

In certain embodiments, the KW-3902 metabolite is selected from the group consisting of 8-(noradamantan-3-yl)-1,3-dipropylxanthine; 1,3-Diallyl-8-(3-noradamantyl)xanthine, 3-allyl-8-(3-noradamantyl)-1-propargylxanthine, 8-(trans-9-hydroxy-3-tricyclo[3.3.1.0^{3,7}]nonyl)-1,3-dipropylxanthine, 8-(cis-9-hydroxy-3-tricyclo[3.3.1.0^{3,7}]nonyl)-1,3-dipropylxanthine, 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine, or a pharmaceutically acceptable salt thereof.

In other embodiments, the xanthine derivative is a xanthine epoxide-derivative compound of Formula II or Formula III, or a pharmaceutically acceptable salt thereof, 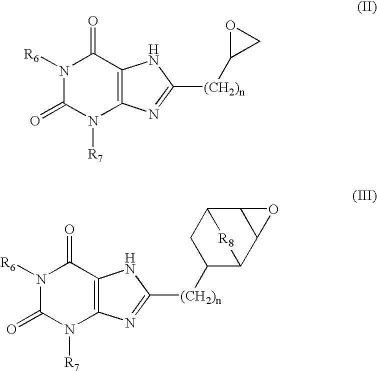
where R₆ and R₇ are the same or different, and can be hydrogen or an alkyl group of 1-4 carbons, R₈ is either oxygen or (CH₂)₁₋₄, and n=0-4.

The xanthine epoxide-derivative compound may be

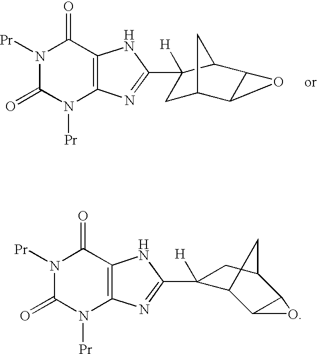

Any amine, hydroxy, or carboxyl side chain on the metabolites, esters, or amides of the above compounds can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

In some embodiments, the administering step comprises administering diuretic therapy and KW-3902 nearly simultaneously. These embodiments include those in which the two compounds are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the patient is directed to take the separate compositions nearly simultaneously, i.e., one injection of one compound is made right after the injection of another compound, etc. In some embodiments, a patient is infused with an intravenous formulation of one compound prior to the infusion of an intravenous formulation of the other compound. In these embodiments, the infusion may take some time, such as a few minutes, a half hour, or an hour, or longer. If the two intravenous infusions are done one right after the other, such administration is considered to be nearly simultaneously within the scope of the present disclosure, even though there was a lapse of some time between the start of one infusion and the start of the next infusion.

In other embodiments the administering step comprises administering either KW-3902 or the diuretic first and then administering the other one of KW-3902 and the diuretic. In these embodiments, the patient may be administered a composition comprising one of the compounds and then at some time, a few minutes or a few hours, later be administered another composition comprising the other one of the compounds. Also included in these embodiments are those in which the patient is administered a composition comprising one of the compounds on a routine or continuous basis while receiving a composition comprising the other compound occasionally. In further embodiments, the patient may receive both compounds on a routine or continuous basis, such a continuous infusion of the compound through an IV line.

In the methods provided herein, the patient can be administered an amount of KW-3902 effective to accelerate removal of excess fluid from the patient in comparison to said diuretic therapy alone. In some embodiments, the amount of KW-3902 is further effective to reduce the term or length-of-stay of the short term hospitalization. In some embodiments, KW-3902 is administered in a dose of at least about 0.5 mg, 1.0 mg, 1.5 mg, 2.0 mg, 2.5 mg, 3 mg, 4 mg, 5 mg, 10 mg, 15 mg, 20 mg, 25 mg, 30 mg, 35 mg, 40 mg, 45 mg, 50 mg, 55 mg, 60 mg, 70 mg, 80 mg, 90 mg, 100 mg, or any number in between. In some embodiments, KW-3902 is administered in a dose lower than 0.5 mg. In other embodiments, KW-3902 is administered in a dose higher than 100 mg.

Preferably, the amount of KW-3902 is effective to reduce the amount of diuretic therapy needed in the individual. In some embodiments, the daily dose of diuretic can be reduced by about 1 mg to about 160 mg. For example, the daily dose of diuretic can be reduced by at least about 1 mg/day, about 5 mg/day, about 10 mg/day, about 15 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 200 mg/day, any number in between or more. Accordingly, in some embodiments, the diuretic is furosemide, and the daily dose of furosemide can be reduced by about 1 mg/day, about 5 mg/day, about 10 mg/day, about 15 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 200 mg/day, or more.

In preferred embodiments, the amount of KW-3902 is effective to reduce the term that the individual is in need of intravenous diuretic therapy. Preferably, the amount of KW-3902 is effective to reduce the term that the individual is in need of intravenous diuretic therapy by at least about 4 hours, 6 hours, 12 hours 18 hours, 24 hours, 32 hours, 48 hours, 60 hours, 72 hours, 84 hours, 96 hours, or 108 hours, or any number of hours in between. In some embodiments, the amount of KW-3902 is effective to reduce the term of intravenous diuretic therapy by more than 108 hours. In preferred embodiments, the amount of KW-3902 is administered to the patient in individual daily doses of about 30 mg.

Preferably, the amount of KW-3902 is effective to reduce the term of short term hospitalization by at least about 4 hours, 6 hours, 12 hours 18 hours, 24 hours, 32 hours, 48 hours, 60 hours, 72 hours, 84 hours, 96 hours, or 108 hours, or any number of hours in between. In some embodiments, the amount of KW-3902 is effective to reduce the term of the short-term hospitalization by more than 108 hours. In preferred embodiments, the amount of KW-3902 is administered to the patient in individual daily doses of about 30 mg.

In some embodiments, the amount of KW-3902 administered to the patient is effective to improve renal function. For example, in some embodiments, the amount of KW-3902 is effective to decrease serum creatinine levels by about 0.01 to about 2.0 mg/dL.

In some embodiments, the amount of KW-3902 administered to the patient is effective to achieve adequate diuresis when administered to the patient receiving diuretic therapy. As used herein the term “adequate diuresis” refers to diuresis sufficient that the patient is no longer in need of intravenous diuretic therapy, as determined using conventional diagnostic methods.

In some embodiments, the administered KW-3902 is in an injectable form, while in other embodiments, the administered KW-3902 is in a solid formulation.

In some embodiments, the diuretic used in the methods provided herein is administered in a dose of about 10 mg, 15 mg, 20 mg, 25 mg, 30 mg, 35 mg, 40 mg, 45 mg, 50 mg, 55 mg, 60 mg, 65 mg, 70 mg, 80 mg, 100 mg, 120 mg, 140 mg, or 160 mg, 180 mg, 200 mg or any number in between. In some embodiments, the diuretic is administered in a dose higher than about 200 mg. In other embodiments, diuretic is administered in a dose less than about 10 mg. In preferred embodiments, the diuretic is administered in a dose of about 40 mg. The intravenous diuretic may be administered as a single injection or as a continuous infusion. When the administration is through a continuous infusion, the dosage of diuretic may be less than 1 mg per hour, 1 mg per hour, 3 mg per hour, 5 mg per hour, 10 mg per hour, 15 mg per hour, 20 mg per hour, 40 mg per hour, 60 mg per hour, 80 mg per hour, 100 mg per hour, 120 mg per hour, 140 mg per hour, or 160 mg per hour, or any number in between. Some embodiments provide that the diuretic is administered in a continuous infusion can be higher than 160 mg per hour.

Preferably, the diuretic administered to the patient is furosemide. Accordingly, in some embodiments, furosemide is administered in a dose of about 10 mg, 15 mg, 20 mg, 25 mg, 30 mg, 35 mg, 40 mg, 45 mg, 50 mg, 55 mg, 60 mg, 65 mg, 70 mg, 80 mg, 100 mg, 120 mg, 140 mg, or 160 mg, 180 mg, 200 mg or any number in between. In most preferred embodiments, furosemide is administered to the patient at a daily dosage of about 40 mg.

In preferred embodiments, the daily dose of the diuretic is decreased over time, following administration of KW-3902 according to the methods provided herein. For example, in some embodiments, the daily dosage of the diuretic can decrease by about 1 mg, 2 mg, 5 mg, 7 mg, 10 mg, 15 mg, 20 mg, 30 mg, 40 mg, 50 mg, 60 mg, 70 mg, 80 mg, 100 mg, or more per day. In some embodiments, the patient can stop receiving daily dosages of diuretics altogether.

In some embodiments provided herein, the subject to be treated by the methods described herein can be administered an AA₁RA, e.g., KW-3902 or a pharmaceutically acceptable salt, ester, amide, metabolite or prodrug thereof in combination with a non adenosine-modifying diuretic, an another compound such as an ACE, an ARB, a beta blocker, an aldosterone inhibitor or other compound or any combination thereof. In some embodiments, the administering step comprises administering said other therapeutic (e.g., ACE, ARB, beta blocker, an aldosterone inhibitor and the like) and said an AA₁RA, e.g., KW-3902 or a pharmaceutically acceptable salt, ester, amide, metabolite or prodrug thereof nearly simultaneously. These embodiments include those in which the AA₁RA, e.g., KW-3902 or a pharmaceutically acceptable salt, ester, amide, metabolite or prodrug thereof and the other therapeutic (e.g., ACE, ARB, beta blocker, an aldosterone inhibitor and the like) are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the subject is directed to take the separate compositions nearly simultaneously, i.e., one pill is taken right after the other or that one injection of one compound is made right after the injection of another compound, etc.

In other embodiments the administering step comprises administering the non adenosine-modifying diuretic, or other therapeutic (e.g., ACE, ARB, beta blocker, an aldosterone inhibitor and the like) first and then administering an AA₁RA, e.g., KW-3902 or a pharmaceutically acceptable salt, ester, amide, metabolite or prodrug thereof. In yet other embodiments, the administering step comprises administering an AA₁RA, e.g., KW-3902 or a pharmaceutically acceptable salt, ester, amide, metabolite or prodrug thereof, first, and then administering the non adenosine-modifying diuretic, or other therapeutic (e.g., ACE, ARB, beta blocker, an aldosterone inhibitor and the like). In these embodiments, the subject may be administered a composition comprising one of the compounds and then at some time, a few minutes or a few hours, later be administered another composition comprising the other one of the compounds. Also included in these embodiments are those in which the subject is administered a composition comprising one of the compounds on a routine or continuous basis while receiving a composition comprising the other compound occasionally.

In some embodiments, the subject can be administered an AA₁RA, e.g., KW-3902 or a pharmaceutically acceptable salt, ester, amide, metabolite or prodrug thereof a non adenosine-modifying diuretic and a beta-blocker. A number of beta-blockers are commercially available. These compounds include, but are not limited to, acebutolol hydrochloride, atenolol, betaxolol hydrochloride, bisoprolol fumarate, carteolol hydrochloride, esmolol hydrochloride, metoprolol, metoprolol tartrate, nadolol, penbutolol sulfate, pindolol, propranolol hydrochloride, succinate, and timolol maleate. Beta-blockers, generally, are beta₁ and/or beta₂ adrenergic receptor blocking agents, which decrease the positive chronotropic, positive inotropic, bronchodilator, and vasodilator responses caused by beta-adrenergic receptor agonists. The embodiments described herein include all beta-blockers now known and all beta-blockers discovered in the future.

In some embodiments provided herein, a subject is administered an AA₁RA, e.g., KW-3902 or a pharmaceutically acceptable salt, ester, amide, metabolite or prodrug thereof, a non adenosine-modifying diuretic and an angiotensin converting enzyme inhibitor or an angiotensin II receptor blocker. A number of ACE inhibitors are commercially available. These compounds, whose chemical structure is somewhat similar, include lisinopril, enalapril, quinapril, ramipril, benazepril, captopril, fosinopril, moexipril, trandolapril, and perindopril. ACE inhibitors, generally, are compounds that inhibit the action of angiotensin converting enzyme, which converts angiotensin I to angiotensin II. The embodiments described herein include all ACE inhibitors now known and all ACE inhibitors discovered in the future.

A number of ARBs are also commercially available or known in the art. These compounds include losartan, irbesartan, candesartan, telmisartan, eposartan, and valsartan. ARBs reduce blood pressure by relaxing blood vessels. This allows better blood flow. ARBs function stems from their ability to block the binding of angiotensin II, which would normally cause vessels to constrict. The embodiments disclosed herein include all ARBs now known and all ARBs discovered in the future.

In some embodiments provided herein, a subject is administered an AA₁RA, e.g., KW-3902 or a pharmaceutically acceptable salt, ester, amide, metabolite, or prodrug thereof, a non adenosine-modifying diuretic and an aldosterone inhibitor. A number of aldosterone inhibitors are commercially available. These compounds include, but are not limited to, spironolactone (ALDACTONE®) and eplerenone (INSPRA®). The embodiments disclosed herein include all aldosterone inhibitors now known and all aldosterone inhibitors discovered in the future.

In still other embodiments, the subject can be administered an AA₁RA, e.g., KW-3902 or a pharmaceutically acceptable salt, ester, amide, metabolite or prodrug thereof, a non adenosine-modifying diuretic and an a prophylactically or therapeutically effective amount of an anticonvulsant. Several anticonvulsants are known in the art and are useful in the compositions and methods described herein. See, e.g., U.S. Patent Application Publication No. 2005/0070524 Extensive listings of anticonvulsants can also be found, e.g., in Goodman and Gilman's “The Pharmaceutical Basis Of Therapeutics”, 8th ed., McGraw-Hill, Inc. (1990), pp. 436-462, and “Remington's Pharmaceutical Sciences”, 17th ed., Mack Publishing Company (1985), pp. 1075-1083, the disclosures of which are hereby expressly incorporated by reference in their entireties. Non-limiting examples of anticonvulsants that can be used in the compositions and methods disclosed hererin include diazepam, midazolam, phenyloin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, and zonisamide, or a pharmaceutically acceptable salt, prodrug, ester, or amide thereof. However, the inclusion of other anticonvulsants, now known or discovered in the future, is within the scope of the present invention.

In some embodiments, an anticonvulsant can be provided in such amount as will be therapeutically or prophylactically effective in the treatment or control of seizures. It will be appreciated that the amount of anticonvulsant contained in an individual dose of each dosage form of the compositions need not in itself constitute an effective prophylactic amount, as the necessary effective amount could be reached by administration of a number of individual doses. Those skilled in the art will appreciate that the amount of anticonvulsant agent present in the compositions and administered to individuals disclosed herein will vary depending upon the age, sex, and bodyweight of the subject to be treated, the particular method and scheduling of administration, and what other anticonvulsant agent, if any is present in the compositions disclosed herein or administered in the methods disclosed herein. Dosage amounts for an individual patient may thus be above or below the typical dosage ranges. Generally speaking, the anticonvulsant agent can be employed in any amount known to be effective at treating, preventing or controlling seizures. The doses may be single doses or multiple doses per day, with the number of doses taken per day and the time allowed between doses varying depending on the individual needs of the patient. Optimization of treatment including dosage amount, method and time of administration can be routinely determined by the skilled practitioner. Specific dosage levels for anticonvulsants that can be used in the pharmaceutical compositions and methods described herein, are included, for example, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosures of which are all hereby expressly incorporated by reference. Representative examples of dosage ranges of anticonvulsants are described below, however, it should be noted that the dosage ranges given below indicate only the typical dosage amounts administered to patients for that particular anticonvulsant agent for the treatment of seizures or epilepsy. Thus they should not be construed as limiting amounts for the purpose of the present invention, as actual therapeutically effective dosage amounts for a patient may be more or less than the exemplary dosage range, depending on the individual.

The compositions and compounds described herein (e.g. diuretics and/or KW-3902) can be administered to a human patient per se, or in pharmaceutical compositions where they are mixed with other active ingredients, as in combination therapy, or suitable carriers or excipient(s). Techniques for formulation and administration of the compositions and compounds (e.g. diuretics and/or KW-3902) described herein may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compositions and/or compounds described herein (e.g. diuretics and/or KW-3902) in a local rather than systemic manner, for example, via injection of the compound directly in the renal or cardiac area, often in a depot or sustained release formulation. Furthermore, one may administer the compositions or compounds in a targeted drug delivery system, for example, in a liposome coated with a tissue-specific antibody. The liposomes will be targeted to and taken up selectively by the organ.

The compositions and compounds described herein (e.g. diuretics and/or KW-3902) can be manufactured in a manner that is itself known, e.g., by means of conventional mixing, dissolving, granulating, dragee-making, levigating, emulsifying, encapsulating, entrapping or tabeleting processes. Compositions and compounds for use in accordance with the methods described herein thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compositions and compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions and compounds provided herein may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for hydrophobic compounds described herein is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds described herein may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Exemplary emulsions used in solubilizing and delivering the xanthine derivatives described above are discussed in U.S. Pat. No. 6,210,687, which is incorporated by reference herein in its entirety, including any drawings.

Many of the compounds used in the methods described herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the methods described herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.01 to 1000 mg/kg of the patient's body weight. The dosage may be a single one or a series of two or more given in the course of one or more days, as is needed by the patient.

The daily dosage regimen of KW-3902 for an adult human patient may be, for example, an oral dose of between 0.1 mg and 500 mg, preferably between 1 mg and 250 mg, e.g., 5 to 200 mg or an intravenous, subcutaneous, or intramuscular dose of between 0.01 mg and 500 mg, preferably between 0.1 mg and 200 mg, e.g., 1 to 100 mg of the compound or a pharmaceutically acceptable salt thereof calculated as the free base, the composition being administered 1 to 4 times per day. Alternatively the compositions of the invention may be administered by continuous intravenous infusion, preferably at a dose of up to 400 mg per day. Thus, the total daily dosage by oral administration will be in the range 1 to 2000 mg and the total daily dosage by parenteral administration will be in the range 0.1 to 400 mg. Suitably the compounds will be administered for a period of continuous therapy, for example for a week or more, or for months or years.

The dose of the intravenous diuretic is that which constitutes standard diuretic therapy. Those of skill in the art know what dosage of diuretics to administer to a patient in need thereof. However, because of the diuretic effect of KW-3902, the need for higher doses of the diuretic are eliminated when KW-3902 is administered to a patient in conjunction with the diuretic. Preferably, the amount of diuretic administered to the patient decreases over time following administration of the AA₁RA. Most preferably, following administration of the AA₁RA, the individual is no longer in need of intravenous diuretic therapy.

Dosage amount and interval may be adjusted individually to provide plasma levels of the active moiety that are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Having now generally described the invention, the same will become better understood by reference to certain specific examples which are included herein for purposes of illustration only and are not intended to be limiting unless other wise specified. All referenced publications and patents are incorporated, in their entirety by reference herein.

### EXAMPLES

### Example 1

A double-blind, randomized multi-center, placebo controlled study was conducted as follows: Approximately 157 subjects were randomized to yield 144 evaluable subjects in an intent-to treat analysis conducted at approximately 50 sites. The study population included males and females at least 18 years of age with New York Heart Association Class II-IV CHF. All subjects had an estimated creatinine clearance between 20 mL/min and 80 mL/min. The average serum creatinine for all individuals at entry was 1.75 mg/dL. All subjects were taking an oral loop diuretic. The demographic data for the study is presented in Table 1 below.

TABLE 1
STUDY DEMOGRAPHICS

| | KW-3902 | | | | |
|---|---|---|---|---|---|
| | Placebo | 2.5 mg | 15 mg | 30 mg | 60 mg |
| n = (ITT population) | 27 | 29 | 30 | 29 | 29 |
| Age (mean yrs) | 67 | 64 | 69 | 66 | 67 |
| Sex (% M/% F) | 74/26 | 66/34 | 65/35 | 70/30 | 69/31 |
| NYHA Class II (%) | 4 | 0 | 0 | 3 | 7 |
| NYHA Class III (%) | 52 | 41 | 58 | 47 | 52 |
| NYHA Class IV (%) | 44 | 59 | 42 | 50 | 41 |

Study visits included pre-treatment days −2 to −1, days 1 to 3 of the Treatment Period, day 4/early Termination and a follow up contact at day 30. Procedures and observations included medical history, physical examination, classification of CHF, vital signs, body weight, CHF signs and symptom scores, Holter monitor recording, chest X-ray, CBC chemistries, creatinine clearance, fluid intake, and urine output.

On treatment days, individuals received KW-3902 intravenously over 120 minutes at one of four doses (2.5 mg, 15 mg, 30 mg, or 60 mg) vs. placebo as both monotherapy and concomitant therapy with diuretics. KW-3902 (or placebo) was administered on days 1 through 3. On day 1, KW-3902 (or placebo) was administered as a monotherapy. 6 hours after administration of KW-3902, IV loop diuretic was given to all treatment groups as needed. On days 2 and 3 KW-3902 was administered as combination therapy with intravenous furosemide, if clinically indicated. Final laboratory data aware collected on day 4 or early termination. A follow-up phone contact was conducted on day 30.

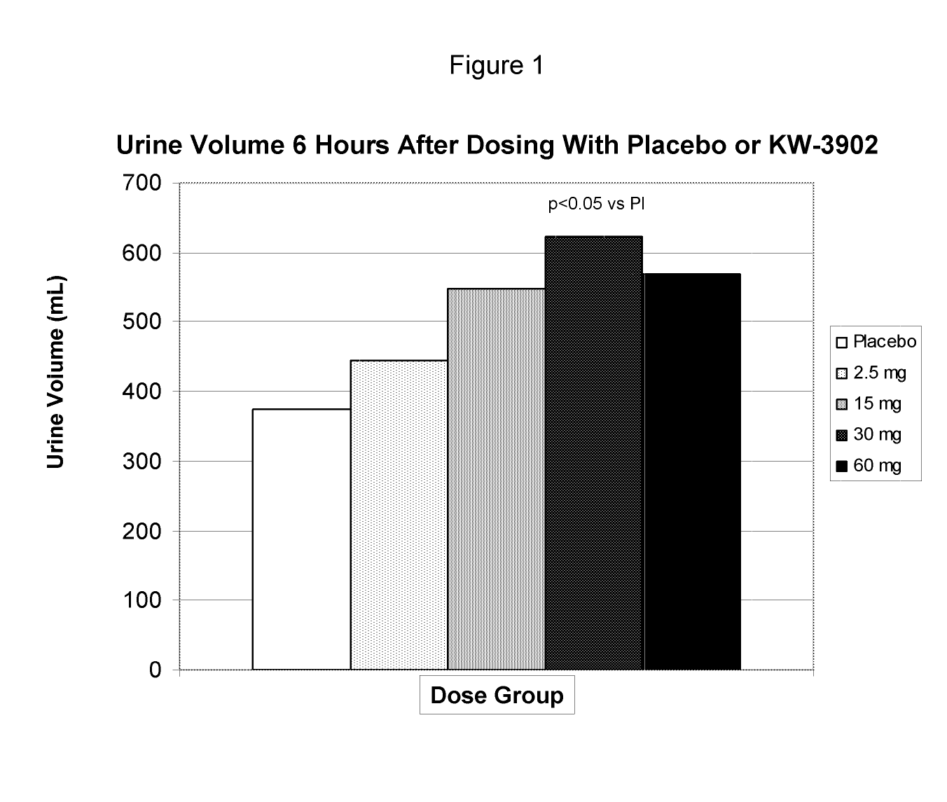

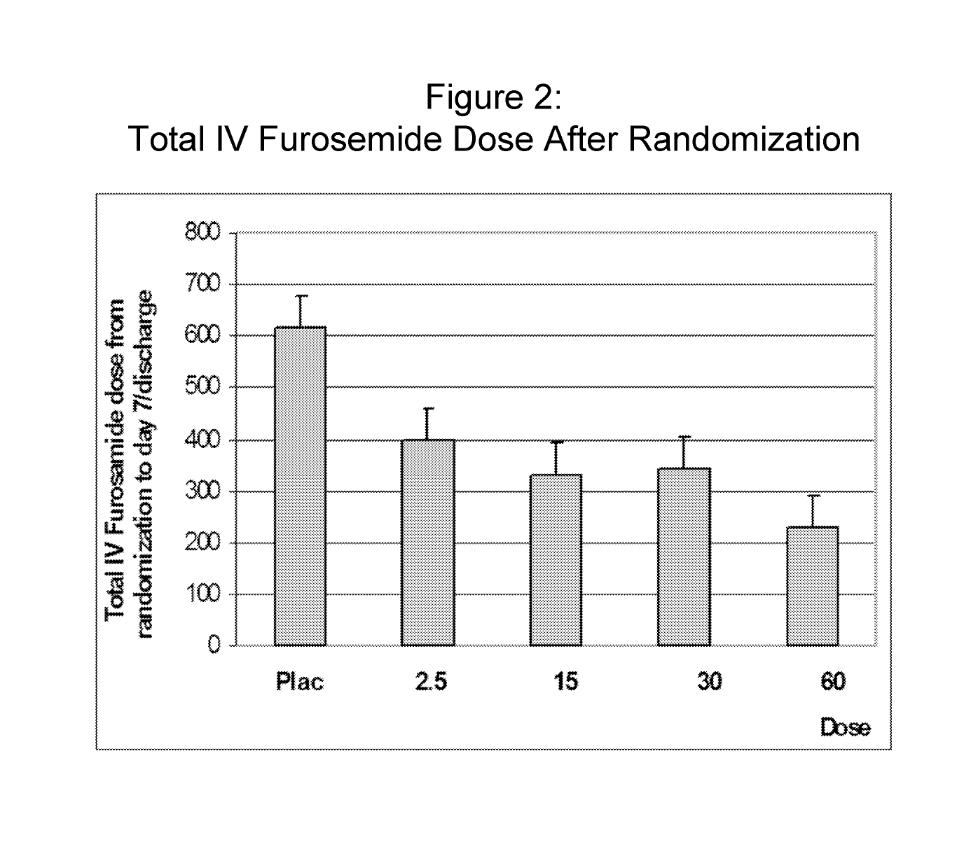

As shown in Table 2 below and FIG. 1, individuals receiving KW-3902 achieved adequate diuresis in less than 4 days and terminated early. As such, KW-3902 resulted in shorter hospitalization and improved treatment time to achieve adequate diuresis. In addition, individuals receiving KW-3902 were administered less furosemide. FIG. 2 depicts the urine output of the individuals treated.

TABLE 2
Premature Termination

| | | | % Early | |
|---|---|---|---|---|
| | | | Termination | |
| | | Adequate | due | Total IV |
| Dose | | Diuresis | to adequate | furosemide |
| Group | n = (ITT pop) | (n = ITT pop) | diuresis | dose (mg) |
| Placebo | 27 | 1 | 4 | 606 |
| 2.5 mg | 29 | 4 | 14 | 397 |
| 15 mg | 31 | 12 | 39 | 331* |
| 30 mg | 30 | 9 | 30 | 342 |
| 60 mg | 29 | 11 | 38 | 229* |

ITT pop = intent to treat population
*p < 0.05

### Example 2

More than 300 subjects hospitalized due to acute CHF requiring intravenous diuretic therapy to treat fluid overload, and presenting with creatinine clearance values between 20 to 80 mL/min were identified. The subjects were randomized to receive either placebo, or 10 mg, 20 mg or 30 mg intravenous KW-3902 per day.

On day 1, KW-3902 (or placebo) was co-administered with intravenous furosemide (LASIX™). The specified dose of KW-3902 (or placebo) was infused over a four hour time period. Subjects received therapy for up to three days. Patients are assessed daily during the initial hospitalization, and at Days 7 ad 14 for signs and symptoms of heart failure. Patients that achieved adequate diuresis were discharged early (“Premature Termination”), and did not receive treatment on Day 2 or Day 3. As shown in Table 2 below, a higher percentage of individuals in the KW-3902 treatment groups were discharged early due as compared to individuals in the placebo treatment group. These data demonstrate that KW-3902 accelerates removal of excess fluid from the patient in comparison to said diuretic therapy alone.

TABLE 3

| | Premature Termination | | | | |
|---|---|---|---|---|---|
| | | | % Early | | % Early |
| | | | Termination | | Termination |
| | | Adequate | Day 2 due | Adequate | Day 3 due |
| Dose | | Diuresis | to adequate | Diuresis | to adequate |
| Group | n | Day 2 (n) | diuresis | Day 3 (n) | diuresis |
| Placebo | 69 | 2 | 4 | 3 | 4.3 |
| 10 mg | 66 | 0 | 14 | 6 | 10.0 |
| KW-3902 | | | | | |
| 20 mg | 70 | 4 | 39 | 6 | 8.5 |
| KW-3902 | | | | | |
| 30 mg | 68 | 3 | 30 | 7 | 10.3 |
| KW-3902 | | | | | |

What is claimed is:

1. A method for treating a patient for acute fluid overload, comprising:
identifying a patient in need of short-term hospitalization to treat acute fluid overload;
hospitalizing the patient;
administering non-adenosine modifying diuretic therapy to the patient while hospitalized; and
administering to the patient an amount of KW-3902 or a pharmaceutically acceptable salt, ester, amide, metabolite or prodrug thereof effective to accelerate removal of excess fluid from the patient in comparison to said diuretic therapy alone.

2. The method of claim 1, wherein the effective amount of KW-3902 or pharmaceutically acceptable salt, ester, amide, metabolite or prodrug thereof is further effective to reduce the term of said short-term hospitalization.

3. The method of claim 2, wherein the daily dose of said non-adenosine modifying diuretic is decreased over time.

4. The method of claim 1, wherein the non adenosine-modifying diuretic is selected from the group consisting of hydrochlorothiazides, furosemide, torsemide, bumetanide, ethacrynic acid, piretanide, spironolactone, triamterene, and amiloridehiazides.

5. The method of claim 4, wherein the diuretic is furosemide.

6. The method of claim 1, wherein said patient has congestive heart failure.

7. The method of claim 1, wherein said effective amount of KW-3902 or pharmaceutically acceptable salt, ester, amide, metabolite, or prodrug thereof is further effective increase the creatinine clearance rate in said subject.

8. The method of claim 1, wherein said effective amount of KW-3902 or pharmaceutically acceptable salt, ester, amide, metabolite, or prodrug thereof is about 2.5 mg, 5 mg, 10 mg, 15 mg, 20 mg, 25 mg, 30 mg, 35 mg, 40 mg, 45 mg, 50 mg, 55 mg, 60 mg, or 70 mg.

9. The method of claim 1, wherein said effective amount of KW-3902 or pharmaceutically acceptable salt, ester, amide, metabolite, or prodrug thereof is 30 mg.

10. The method of claim 1, wherein said subject exhibits a creatinine clearance rate of about 20 to 80 mL/min.

11. A method of reducing the treatment time to achieve adequate diuresis in a patient experiencing acute fluid overload or congestive heart failure, comprising:
identifying a patient experiencing acute fluid overload or congestive heart failure; and
administering to said patient a therapeutically effective amount of KW-3902 or a pharmaceutically acceptable salt, ester, amide, prodrug, or metabolite thereof.

12. The method of claim 11, wherein the effective amount of KW-3902 or pharmaceutically acceptable salt, ester, amide, metabolite or prodrug thereof is further effective to reduce the term of said short-term hospitalization.

13. The method of claim 12, wherein the daily dose of said diuretic is decreased over time.

14. The method of claim 11, wherein the non adenosine-modifying diuretic is selected from the group consisting of hydrochlorothiazides, furosemide, torsemide, bumetanide, ethacrynic acid, piretanide, spironolactone, triamterene, and amiloridehiazides.

15. The method of claim 14, wherein the diuretic is furosemide.

16. The method of claim 11, wherein said patient has congestive heart failure.

17. The method of claim 11, wherein said effective amount of KW-3902 or pharmaceutically acceptable salt, ester, amide, metabolite, or prodrug thereof is further effective to increase the creatinine clearance rate in said subject.

18. The method of claim 11, wherein said effective amount of KW-3902 or pharmaceutically acceptable salt, ester, amide, metabolite, or prodrug thereof is about 2.5 mg, 5 mg, 10 mg, 15 mg, 20 mg, 25 mg, 30 mg, 35 mg, 40 mg, 45 mg, 50 mg, 55 mg, 60 mg, or 70 mg.

19. The method of claim 11, wherein said effective amount of KW-3902 or pharmaceutically acceptable salt, ester, amide, metabolite, or prodrug thereof is 30 mg.

20. The method of claim 1, wherein said patient is also receiving a therapeutic selected from the group consisting of an angiotensin II converting enzyme inhibitor (ACE inhibitor), an angiotensin receptor blocker (ARB), a beta blocker, and an aldosterone inhibitor.

21. The method of claim 11, wherein said patient is also receiving a therapeutic selected from the group consisting of an angiotensin II converting enzyme inhibitor (ACE inhibitor), an angiotensin receptor blocker (ARB), a beta blocker, and an aldosterone inhibitor.

22. The method of claim 1, wherein said patient also receives an anticonvulsant.

23. The method of claim 11, wherein said patient also receives an anticonvulsant.
